(19) United States
(12) Patent Application Publication
(10) Pub. No.: US 2021/0008305 A1
(43) Pub. Date: Jan. 14, 2021

(54) INDUCTION SPACER FOR INHALER

(71) Applicant: Health Research, Inc., Buffalo, NY (US)

(72) Inventor: Khurshid GURU, East Amherst, NY (US)

(21) Appl. No.: 16/979,464

(22) PCT Filed: Mar. 11, 2019
(86) PCT No.: PCT/US19/21700

§ 371 Date: Sep. 9, 2020

Related U.S. Application Data
(60) Provisional application No. 62/640,710, filed on Mar. 9, 2018.

(51) Int. Cl. A61M 15/00; A61M 16/06

(52) U.S. Cl. CPC A61M 15/0086; A61M 15/009; A61M 2205/8281; A61M 15/0021; A61M 16/06

(57) ABSTRACT

A spacer for an inhaler includes a housing defining a chamber. The housing has an ambient port in fluid communication with ambient air, an inhaler port configured for attachment of an inhaler, and an outlet. The spacer further includes a fan for generating a flow of air through the chamber from the ambient port to the outlet. The fan may be powered by way of a spring such that the fan generates the flow of air as stored energy is released from a spring. Embodiments of the spacer may have an actuator for triggering a release of stored energy from the spring thereby actuating the fan. A mask or mouthpiece may be provided at the outlet.

### CROSS-REFERENCE TO RELATED APPLICATIONS

This application claims priority to U.S. Provisional Application No. 62/640,710, filed on Mar. 9, 2018, the disclosure of which is incorporated herein by reference.

### FIELD OF THE DISCLOSURE

The present disclosure relates to medicinal inhalers, and in particular, to spacers coupled to such inhalers.

### BACKGROUND OF THE DISCLOSURE

Metered-dose inhalers (MDIs) are commonly used by individuals needing medications delivered into the body via the lungs. Such medications may treat conditions like asthma or chronic obstructive pulmonary disease (COPD). However, the use of inhalers can be difficult for some individuals because the metered dose must be timed with deep inhalation in order to minimize wasted medication that does not reach the individual's lungs.

Small volume spacers have been developed to improve the effectiveness of inhalers by reducing wasted medication in a dose. The spacers provide a chamber into which the inhaler deposits the medicament. In the chamber, the medicament is mixed with a small volume of ambient air and the mixture is contained in the chamber, ready for the individual upon inhalation. In this way, the timing of the metered does is not as closely tied to the inhalation of the individual. Additionally, medicament that is exhaled can be at least partially re-inhaled in one or more subsequent breaths.

However, such devices remain dependent upon the compliance of the user. Some individuals, especially infants and young children, may not be sufficiently able to comply with the actions necessary for the use of inhalers with or without small volume spacers. For example, infants often cry in distress at the very times when medication would be useful in resolving the distress. Accordingly, there remains a need for a device to enhance the performance of inhalers for use by individuals, and especially non-compliant individuals.

### BRIEF SUMMARY OF THE DISCLOSURE

The present disclosure may be embodied as a spacer for an inhaler. The spacer includes a housing defining a chamber. The housing has an ambient port in fluid communication with ambient air, an inhaler port configured for attachment of an inhaler, and an outlet. The spacer further includes a fan for generating a flow of air through the chamber from the ambient port to the outlet. The spacer includes a primer that is operable by a user to provide energy to power the fan. In some embodiments, the fan is powered by way of a spring such that as stored energy is released from a spring, the fan generates a flow of air. In some embodiments, the fan is located between the ambient port and the inhaler port. In other embodiments, the fan is located between the inhaler port and the outlet.

The spacer also has an actuator for triggering a release of stored energy from the spring thereby actuating the fan. In some embodiments, the actuator is further configured to actuate the inhaler such that a dose of medicament is released from the inhaler into the flow of air. The spacer may further include a primer for loading the spring of the fan. The primer may be, for example, a lever, a rotatable knob, etc. One or more components, such as, for example, a mask, mouthpiece, etc., may be coupled to the outlet. The component(s) may be configured to be releasably coupled. For example, the outlet may be a bayonet mount, and the coupled component (e.g., mask, mouthpiece, etc.) may have a corresponding bayonet mount to cooperate with the outlet.

### DESCRIPTION OF THE DRAWINGS

For a fuller understanding of the nature and objects of the disclosure, reference should be made to the following detailed description taken in conjunction with the accompanying drawings, in which:

FIG. 1 is a perspective view diagram of a device according to an embodiment of the present disclosure, shown with an inhaler;

FIG. 2A is a perspective view of another embodiment of the present disclosure having a mask;

FIG. 2B shows the spacer of FIG. 2A showing the mask coupled to the outlet;

FIG. 3A is a perspective view of a device according to another embodiment of the present disclosure;

FIG. 3B is another perspective view of the device of FIG. 3A;

FIG. 4A is an exploded view diagram of the device of FIGS. 3A and 3B; and

FIG. 4B is another exploded view diagram of the device of FIGS. 3A, 3B, and 4A.

### DETAILED DESCRIPTION OF THE DISCLOSURE

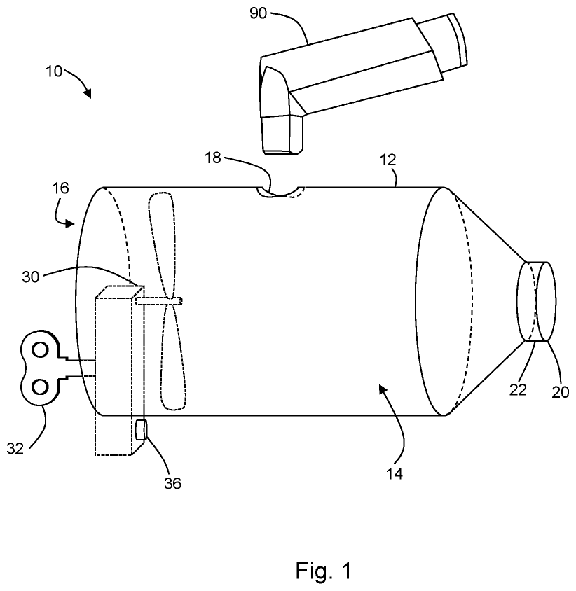

With reference to FIG. 1, the present disclosure may be embodied as a spacer 10 for an inhaler 90, for example, a metered-dose inhaler (MDI). It is noted that, in some embodiments, the inhaler 90 is not a part of the spacer 10—e.g., is not included with the spacer. In such embodiments, the inhaler 90 is an environmental component suitable for attachment to the inhaler 10. The spacer 10 includes a housing 12 which defines a chamber 14an interior volume. The housing 12 includes an ambient port 16 for introducing ambient air into the chamber 14. In this way, the chamber 14 is in fluid communication with the ambient environment by way of the ambient port 16. An inhaler port 18 is configured to be attached to an inhaler 90. An inhaler 90 may be attached to the inhaler port 18 either directly or indirectly. For example, the inhaler 90 may be (indirectly) attached to the inhaler port 18 by way of a conduit. The inhaler port 18 is configured such that, when an inhaler 90 is connected thereto, a dose of medicament from the inhaler 90 will be expelled into the chamber 14.

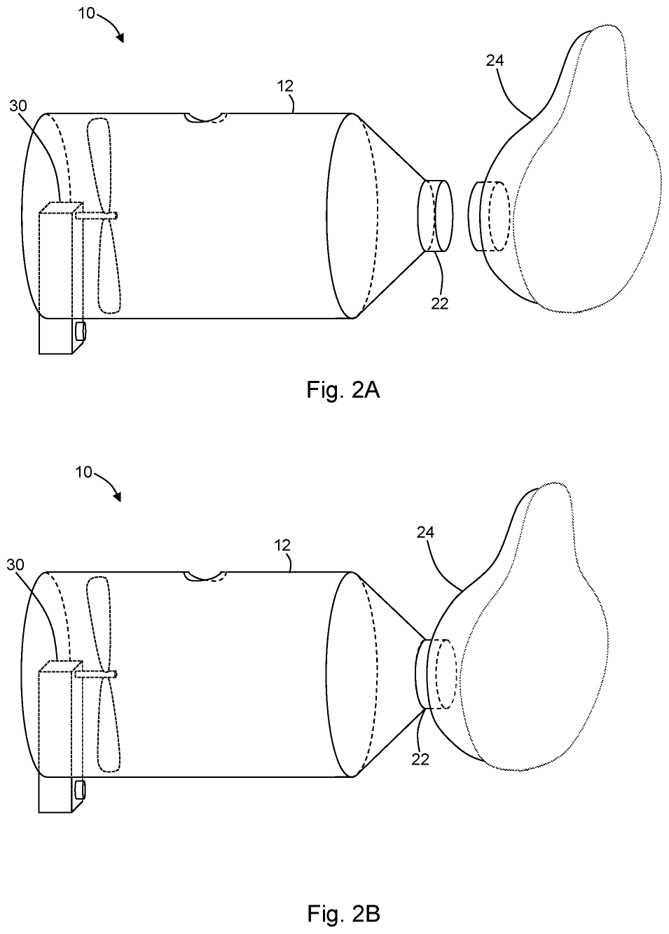

The housing 12 also includes an outlet 20 configured to be in fluid communication with a user's lungs when the spacer 10 is used. The outlet 20 is configured to provide air and medicament to a user and may be considered a user interface. The outlet 20 may include a coupler 22 for connection to one or more attachments. For example, the coupler 22 may be a bayonet coupler, a threaded coupler, etc. The spacer 10 may include an oronasal mask 24 (FIG. 2) coupled to the outlet 20 of the housing 12. The mask 24 may be coupled by way of a coupler 22. In some embodiments, the mask 24 is releasably coupled so that the mask 24 can be selectively attached or detached. In other embodiments, the mask 24 is not releasable. In other embodiments, the mask makes up a part of the outlet 20 itself. In some embodiments, the spacer 10 may include a mouthpiece such that only the user's mouth is involved, rather than both the mouth and nose. In light of the present disclosure, it will be apparent that other components may be attached to the outlet 20.

The spacer 10 further includes a fan 30 for generating a flow of air. The fan 30 is configured to generate a flow of air through the chamber 14 from the ambient port 16 to the outlet 20. In some embodiments, the fan 30 may be positioned upstream from the inhaler port 18 with respect to the generated air flow (e.g., between the ambient port 16 and the inhaler port 18) or downstream from the inhaler port 18 (e.g., between the inhaler port 18 and the outlet 20).

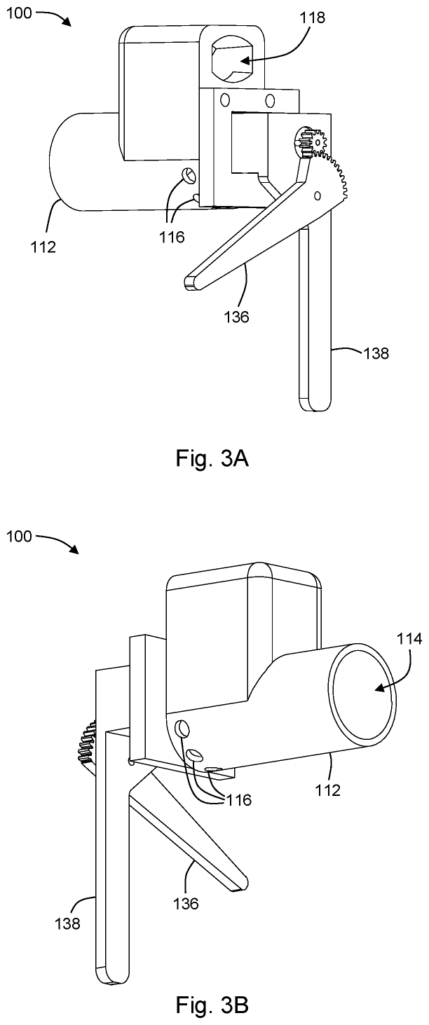

The spacer 10 further includes a primer 32 such as a winding key (FIGS. 1 and 2), a lever (FIGS. 3A-3B), or other suitable primer. The primer 32 is operable by an operator to provide power to the fan 30. For example, the primer 32 may be operated by an adult operator while the spacer 10 is being used to provide medicament to a child (i.e., at the outlet 20). In other embodiments, the user is also the operator and can power the fan while using the spacer 10. The primer 32 can be of any configuration useful to for manual operation, such as a winding key, a rotatable knob, a pull-string, a lever, a trigger, or any other suitable mechanism.

In some embodiments, the fan 30 may be powered by an energy store, for example, a mechanical energy store such as a spring. The primer 32 may be operable to provide power to the fan 30 by loading the spring with energy. In this way, the fan 30 will generate a flow of air when stored energy is released from the spring. In embodiments using a spring as a mechanical energy store, the spring component may be of any suitable design. Such designs are known in the art for such uses and utilize mechanisms having, for example, wound springs, torsion springs, etc.

An actuator 36 may be provided for triggering a release of energy stored in the spring, thereby actuating the fan 30. The actuator 36 may be a push button, a pull trigger, a clip, or any other configuration suitable for the disclosed uses as will be apparent in light of the present disclosure. In some embodiments, the actuator 36 is also configured to actuate the inhaler 90 (when an inhaler 90 has been attached to the spacer 10). In this way, the inhaler 90 will release a dose of medicament into the flow of air caused by the fan 30. In such embodiments, the timing of the inhaler 90 actuation relative to the fan 30 actuation may be staggered such that a dose is delivered before, simultaneous with, or after the fan 30 actuation.

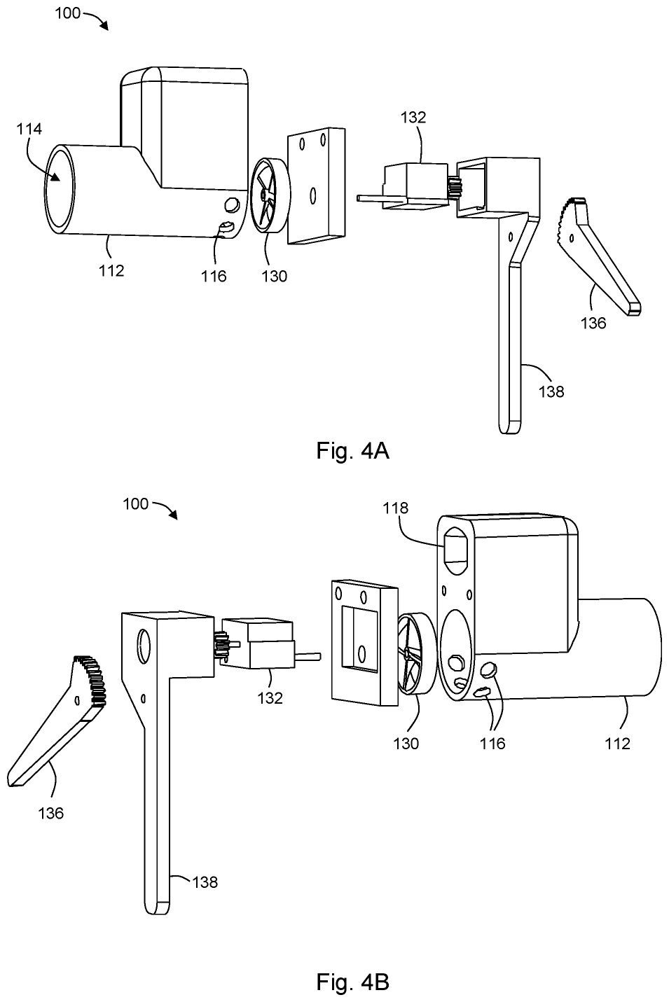

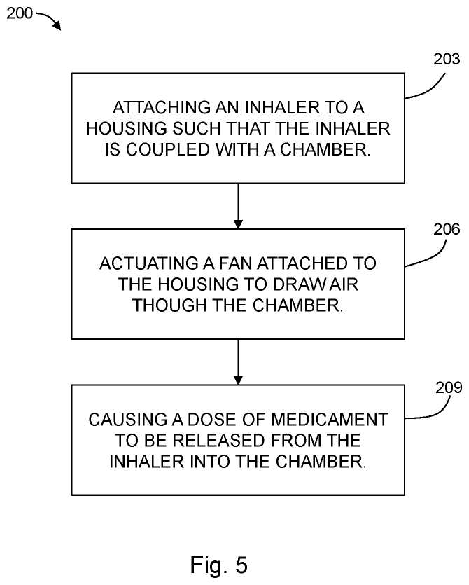

FIGS. 3A, 3B, 4A, and 4B show another embodiment of a spacer 100 having a housing 112 with multiple ambient ports 116 for instroducing air into a chamber 114. An inhaler port 118 allows for fluid communication with an inhaler (not shown). In the spacer 100, a fan 130 is powered by way of a gearbox 132, which is in turned powered by a primer 134. In the device 100 depicted in the present embodiment, the primer 134 is a lever 136 which is squeezed towards a handle 138. The gearbox 132 may additionally include a spring or other energy store. Such a primer 134 may be used to power the fan directly or may be used to store energy which can then be initiated using an actuator as described above.

In another aspect, the present disclosure may be embodied as a method 200 for providing aerosolized medicament. An inhaler is attached 203 to a housing such that the inhaler operably coupled with a chamber of the housing. The method 200 includes actuating 206 a fan attached to the housing so as to cause ambient air to be drawn through the chamber to an outlet. A dose of medicament is caused 209 to be released from the inhaler into the chamber. In this way, the dose of medicament is mixed with the air drawn through the chamber by the fan.

Although the present disclosure has been described with respect to one or more particular embodiments, it will be understood that other embodiments of the present disclosure may be made without departing from the spirit and scope of the present disclosure.

We claim:

1. A spacer for an inhaler, comprising:
a housing defining a chamber, the housing having an ambient port in fluid communication with ambient air, an inhaler port configured for attachment of an inhaler, and an outlet;
a fan for generating a flow of air through the chamber from the ambient port to the outlet; and
a primer operable by a user to provide energy for powering the fan.

2. The spacer of claim 1, wherein the fan is powered by way of a spring such that the fan generates the flow of air as stored energy is released from a spring, and further comprising an actuator for triggering a release of stored energy from the spring thereby actuating the fan.

3. The spacer of claim 2, wherein the actuator is further configured to actuate the inhaler such that a dose of medicament is released from the inhaler into the flow of air.

4. The spacer of claim 1, wherein the fan is located between the ambient port and the inhaler port.

5. The spacer of claim 1, wherein the fan is located between the inhaler port and the outlet.

6. The spacer of claim 1, wherein the primer is a lever.

7. The spacer of claim 1, wherein the primer is a rotatable knob.

8. The spacer of claim 1, further comprising an oronasal mask coupled to the outlet of the housing.

9. The spacer of claim 8, wherein the mask is releasably coupled to the outlet.

10. The spacer of claim 8, further comprising a mouthpiece coupled to the outlet of the housing.

11. The spacer of claim 10, wherein the mouthpiece is releasably coupled to the outlet.

12. A method for providing aerosolized medicament, the method comprising:
attaching an inhaler to a housing such that the inhaler operably coupled with a chamber of the housing;
actuating a fan attached to the housing so as to cause ambient air to be drawn through the chamber to an outlet; and
causing a dose of medicament to be released from the inhaler into the chamber.
